(19) United States
(12) Patent Application Publication
(10) Pub. No.: US 2024/0207851 A1
(43) Pub. Date: Jun. 27, 2024

(54) METHOD FOR AUTOMATIC ONLINE DETECTION OF AT LEAST ONE BIOLOGICAL TARGET SUBSTANCE IN A LIQUID AND ONLINE ANALYZER

(71) Applicant: IST Innuscreen GmbH, Berlin (DE)

(72) Inventor: Timo Hillebrand, Hönow (DE)

(21) Appl. No.: 18/555,894

(22) PCT Filed: Apr. 8, 2022
(86) PCT No.: PCT/EP2022/059368

§ 371 Date: Oct. 18, 2023

(30) Foreign Application Priority Data
Apr. 19, 2021 (DE) 10 2021 109 852.1

(51) Int. Cl. B01L 3/00; C12N 15/10; C12Q 1/6825; C12Q 1/686

(52) U.S. Cl. CPC B01L 3/502761; B01L 3/502715; C12N 15/1013; C12Q 1/6825; C12Q 1/686; B01L 2200/04; B01L 2200/16; B01L 2300/047; B01L 2300/0663; B01L 2400/0487

(57) ABSTRACT

A method for automated online detection of at least one biological target substance in a liquid using an online analyzer includes: transporting a first volume of liquid into a reaction chamber; concentrating biomolecules in the first volume of liquid by binding to a plurality of particles in the reaction chamber; transporting the particles with the bound biomolecules into a detection unit having a first microfluidics unit and a second microfluidics unit fluidically connectable thereto; releasing and/or isolating nucleic acids from the biomolecules bound on the particles; transporting an eluate of the released and/or isolated nucleic acids into the second microfluidics unit; amplifying a target nucleic acid in the second microfluidics unit; registering a measurement signal representing progress of the amplifying and/or on a number of copies of the target nucleic acid, and determining of the target substance in the sample based on the registered measurement signal.

The invention relates to a method for automated online detection of at least one biological target substance in a liquid and to an online analyzer, which is embodied for automated execution of the method.

The detection of biological parameters plays an increasingly important role in the process industry, for example, in the monitoring and cleaning of industrial water or clear water, in food production and in the pharma and life science fields, for example, in the monitoring of biotechnological processes performed in fermenters. Target substances to be detected or monitored can be, for example, viruses, bacteria as well as plasmid-associated, bacterial resistance genes.

The detection of such biological target substances in liquids occurs using molecular genetic methods, preferably by means of amplification techniques such as PCR or real time PCR. For this, samples of the liquid to be analyzed are taken and analyzed in specialized laboratories, which have the necessary devices and systems. For automated sample taking from processes, e.g., from clarification basins, fermenters, production vessels or lines, manual and automated sample taking apparatuses and sample collectors are known. The collected samples are, as a rule, sent to a laboratory for detailed analysis, where the extraction of sample nucleic acids and the specific and, in given cases, quantitative detection of the biological target substance is performed manually or at least partially automatically.

Especially in the field of water and wastewater analysis, online analyzers are known, which perform sample taking and quantitative determination of a, most often, inorganic analyte in the sample completely automatically. Such online analysis can be performed continuously or discontinuously.

Known from US 2015/0224502 A1 is a sample collecting device for sample taking from bodies of water. The device uses a plurality of sample cartridges, which are adapted to receive water from the body of water to be monitored and, in given cases, to retain samples of components of the liquid or of solids in a filter or adsorption medium, while the collected water is returned from the cartridges into the body of water. The samples can either be stored for later laboratory analysis or analyzed directly in the device. For this, for example, biological substances retained by the filter or adsorption medium can be released as lysate and sent to an analysis module for further analysis, e.g., by means of qPCR.

In the case of small concentrations of the target substances in the liquid to be analyzed, there is the problem of obtaining in the sample taking a sufficient amount of the target substance for the subsequent analysis. In the case of a sample taking as in US 2015/0224502 A1, where water moves through a sample collector, even dissolved substances or solids present in small concentrations in water can be accumulated in the filter medium in sufficient quantity by passing a correspondingly large water volume through the sample collector. Such is, however, only practical, when the liquid to be analyzed can be returned without limitation back into the medium after the sampling, such as is the case for, e.g., bodies of water. In the monitoring of processes, e.g., in the upgrading of water, in the foods industry or in the pharma field, such a reintroduction of the removed liquid is often not possible. In such case, there is a need for a method for efficient concentrating of sample components to be detected, while keeping the liquid volume removed from the process for the analysis small.

Another disadvantage of the device described in US 2015/0224502 A1 is that the substance retained in the filter or adsorption medium is extracted by lysis and elution from the filter or adsorption medium into the analysis module. In such case, it is, on the one hand, to be expected that only a relatively small part of the target substance actually gets from the filter or adsorption medium into the analysis module, while, on the other hand, a certain minimum volume of liquid is required for rinsing the filter, such that the solution reaching the analysis module has a relatively large volume coupled with a correspondingly small concentration of the target substance.

An object of the invention is to provide improved method for automated online detection of a biological target substance in a liquid and online analyzer suitable for performing the method. Especially, the method and the apparatus should enable an efficient concentrating of the target substance when sample taking and a low-loss forwarding of the concentrated target substance into a following analytical unit.

The object is achieved by the method as defined in claim 1 and the apparatus as defined in claim 10. Advantageous embodiments are set forth in the dependent claims and in the following description.

The method of the invention for automated online detection of at least one biological target substance in a liquid, especially water, e.g., wastewater, by means of an online analyzer, comprises steps as follows:
transporting a predetermined first volume of liquid via a sample supply line into a reaction chamber of a process unit of the online analyzer;
concentrating biomolecules contained in the first volume of liquid by binding to a plurality of particles in the reaction chamber;
transporting the particles with the bound biomolecules into a detection unit of the online analyzer, wherein the detection unit has a first microfluidics unit and a second microfluidics unit fluidically connectable with the first microfluidics unit;
releasing and/or isolating nucleic acids from the biomolecules bound on the particles;
transporting an eluate comprising the released and/or isolated nucleic acids into the second microfluidics unit;
amplifying a target nucleic acid in the second microfluidics unit;
registering a measurement signal, which represents a measured variable dependent on progress of the amplifying and/or on a number of copies of the target nucleic acid in the second microfluidics unit, and
qualitative and/or quantitative determining of the target substance in the sample based on the registered measurement signal by a control electronics of the analyzer.

The terminology, biomolecules, means, here and in the following, bacteria, bacteriophages, viruses, subcellular particles and released nucleic acids or nucleic acid fragments. The target nucleic acid amplified in the second microfluidic unit can be either the target substance to be detected or a nucleic acid of the target substance to be detected.

The first and second microfluidics units can be accommodated in two separate cartridges or in a shared cartridge.

Since the biomolecules are bound on particles and transported with such into a first microfluidics unit, in order in the first microfluidics unit to release and/or isolate nucleic acids for further analysis, a relatively high fraction of the biomolecules won from the first volume of liquid can be concentrated in a small eluate volume compared with the method described in the state of the art, in the case of which biomolecules after binding to a filter medium are released from the filter medium by a rinsing procedure. Thus, the method of the invention effects not only an efficient concentrating of the target substance to be determined but also enables in the case of a low concentration of the target substance in the investigated liquid use of a comparatively small first volume of liquid to be removed from the process. As a result, the method is universally suitable for monitoring liquids in a large number of different industrial processes, independently of whether the first volume transported into the process unit must after the concentrating be thrown away or, in given cases, disposed of, or whether it can be returned into the process.

The sample supply line can in a possible embodiment of the method be fluidically connected with a sample supply. The sample supply can contain a supply of the investigated liquid. It can be connected with a sample taking apparatus, which is adapted, event driven or with a predetermined sampling frequency to withdraw liquid from a body of water, from a liquid conveying line of a supply network, or from a process container, e.g., a fermenter, and to transport such into the sample supply. Alternatively, the sample taking apparatus can also be connected directly with the sample supply line of the online analyzer. The transport of liquid into the process unit of the online analyzer can be controlled automatically by the control electronics of the analyzer.

After the concentrating of biomolecules contained in the first volume of liquid, the first volume of liquid can be removed from the reaction chamber and a second volume of the liquid introduced into the reaction chamber. Biomolecules contained in the second volume of liquid can then be concentrated in the reaction chamber by binding to the particles remaining in the reaction chamber. These steps can be repeated multiple times, in order, in this way, cumulatively to increase the amount of biomolecules bound on the particles. In this way, the sensitivity of the method can be increased.

The particles can be magnetic or paramagnetic particles, to which the biomolecules bind, especially unselectively.

The concentrating of biomolecules contained in the liquid can include the introduction of one or more reaction components into the reaction chamber of the process unit. The reaction components in the reaction chamber can comprise, for example, an alginate solution and a salt of a divalent or polyvalent cation, or of an acid, such that an alginate gel-biomolecule complex forms on the particles. The particles can be introduced into the reaction chamber at the same time as the reaction components or be provided therein before or after transport of the first volume of liquid into the reaction chamber.

The transporting of the particles with the bound biomolecules into the detection unit in a possible method embodiment comprises at least steps as follows:
transporting the particles by means of a magnet, especially a magnet movable relative to the reaction chamber, by means of a pin, by means of a magnetic pin and/or by means of a pipetting apparatus into a region of the reaction chamber where a fluid line connects the reaction chamber fluidically with the first microfluidics unit, and
rinsing the particles through the fluid line into the first microfluidics unit.

For transporting the particles, one of the described options of particle transfer (magnet, pin, etc.) can be used, or different options can be combined. Thus, in a first example, it can be provided that the particles are moved first by means of the magnet (for example, in the direction of a port, via which the particles can be removed) and then transferred by means of the pin, the magnetic pin or the pipetting apparatus into the said region of the reaction chamber. In a second example, the reversed sequence can be provided, such that the particles are transferred first by means of the pin, the magnetic pin or the pipetting apparatus and then moved by means of the magnet.

The magnet can be a permanent magnet or an electromagnet. The magnetic part of the magnetic pin can likewise be embodied as a permanent magnet or as an electromagnet.

The rinsing of the particles into the first microfluidics unit can occur by introducing a small volume fraction of the liquid contained in the reaction chamber. The transport of the liquid and the particles can be effected by action of the force of gravity, e.g., a hydrostatic pressure of the liquid in the reaction chamber, in that the region, in which the fluid line connects with the reaction chamber lies somewhat depressed relative to the floor of the reaction chamber. After the particles are transferred into the detection unit, the residual liquid in the reaction chamber can be drained from the reaction chamber via a suitable drain. Alternatively, most of the liquid can first be drained from the reaction chamber, while the particles are still retained in the reaction chamber by means of the magnet. The particles can then be rinsed with the remainder of the liquid into the detection unit, thus into the first microfluidics unit.

The releasing and/or isolating of nucleic acids from the biomolecules bound on the particles in the first microfluidics unit can comprise at least steps as follows:
contacting the particles with the bound biomolecules with one or more lysis reagents, in order to release nucleic acids of the adsorbed biomolecules;
binding the released nucleic acids on the particles; and
washing the particles with the bound nucleic acids with a wash solution.

In this embodiment of the method, the same particles serve, advantageously, thus, not only for extraction and concentrating of the biomolecules from the liquid to be monitored, but, instead, also for binding and collecting of released nucleic acids.

Furthermore, the method can comprise the releasing of the nucleic acids from the particles by eluting and
transporting an eluate comprising the nucleic acids into the second microfluidics unit for subsequent amplification.

In an alternative embodiment, the releasing and/or isolating of nucleic acids from the biomolecules bound on the particles in the first microfluidics unit can comprise steps as follows:
thermal releasing of the biomolecules (e.g., free DNA) bound on the particles, or of nucleic acid contained in the biomolecules (e.g., bacteria), in a buffer solution, for example, in a phosphate buffered, salt solution, in water or in a tris buffer, which optionally includes a complexing reagent for complexing divalent or polyvalent cations bound on the particles with the biomolecules, and
transporting the buffer solution with the biomolecules and nucleic acids dissolved therein into the second microfluidics unit for subsequent amplification.

The thermal releasing can comprise a dispersing of the particles in the buffered solution, in given cases, containing the complex former, and an incubation. In such case, the alginate-gel structure present on the particles surfaces is dissolved and the biomolecules bound therein are dissolved. This method variant is suited, for example, for applications, in the case of which the target substance to be determined is a nucleic acid, which is already present released in the liquid to be analyzed. Thus, in the case of this alternative method embodiment, a classic lysis can be omitted. The nucleic acids bound on the particles in an alginate-complex can, in contrast, only be brought into solution by addition of the buffer solution to the particles, in given cases, with further addition of the complexing reagent, e.g., a chelate former such as EDTA. It is also possible to apply this method variant, when the biomolecule target substance is a bacterium or a virus. In such case, the biomolecules bound on the particles can be thermally a destroyed and the contained nucleic acid released.

The amplification executed in the second microfluidics unit can be performed, for example, by means of conventional PCR based methods. The qualitative or quantitative determining of the target substance can, in likewise conventional manner, be performed by registering by means of a sensor at a certain point in time or at a plurality of points in time, in each case, at least one measured value of a measured variable, which correlates with the number of copies of a target nucleic acid produced by the amplification and present at the particular point in time. From the obtained measured value or measured values as a function of time, the presence of the target nucleic acid and therewith the biological target substance in the liquid to be monitored can be qualitatively detected or a quantitative value, e.g., a concentration, of the biological target substance in the liquid can be ascertained and output by the control electronics as a measurement result.

The control electronics of the online analyzer advantageously executes all described method steps automatically. For this, it can, for example, effect a liquid transport through actuation of controllable valves and/or pumps or a relative movement of the magnet relative to the reaction chamber by a drive moving the magnet and/or the reaction chamber.

The invention resides also in an online analyzer for detection of at least one biological target substance in a liquid, especially by applying the above-described method. The online analyzer comprises at least the following components:
a control electronics, a process unit and a detection unit,
wherein the process unit includes at least one reaction chamber and a sample supply line connected with the reaction chamber, and
wherein the process unit is adapted to receive a predetermined volume of liquid in the reaction chamber and to concentrate biomolecules contained in the liquid by binding to particles; and
wherein the detection unit has:
   fluidically connectable with the reaction chamber, a first microfluidics unit, which is adapted to release and/or to isolate nucleic acids from the biomolecules bound on the particles;
   connected with the first microfluidics unit, a second microfluidics unit, which is adapted to receive an eluate comprising the released and/or isolated nucleic acids and to amplify a target nucleic acid; and
   a sensor (especially an optical sensor), which is adapted to produce a measurement signal dependent on progress of an amplification performed in the second microfluidics unit and/or on a number of copies of the target nucleic acid in the second microfluidics unit and to output such to the control electronics; and
wherein the control electronics is adapted, based on the measurement signal output from the sensor, qualitatively and/or quantitatively to determine the biological target substance in the liquid.

The analyzer can further have a sample pump, wherein the control electronics is adapted to control the sample pump for transporting a predetermined volume of sample into the reaction chamber. The sample supply line can, such as already mentioned, be connectable fluidically with a sample supply or directly with a body of water or a line in a process plant, a fermenter or other process container.

The analyzer can further comprise at least one reaction component reservoir, which contains at least one reaction component for the concentrating of the biomolecules in the reaction chamber. The analyzer can provide, for example, an alginate solution, a solution containing a salt of a divalent or polyvalent cation, or, alternatively, a weak acid as reaction components in one or more separate reservoirs. The reaction component reservoir can be a container, which is connectable fluidically with the reaction chamber via a reaction components supply line, for example, one having a selectively closable/openable valve. The particles can be provided in the reaction chamber or likewise added from a reaction component reservoir as reaction components to the liquid transported into the reaction chamber for the concentrating of the biomolecules. The particles can be magnetic and/or paramagnetic particles. The control electronics of the analyzer can advantageously be adapted to dose, or meter, a predeterminable amount of the at least one reaction component, and all reaction components, into the reaction chamber. For this, one or more pumps controlled by the control electronics can be provided for the transport of the at least one reaction component. Additionally or alternatively, the control electronics can control the valve.

The analyzer can further comprise means for moving, especially stirring, the liquid in the reaction chamber. The means can comprise, e.g., a stirrer, a supply line for introducing inert gas into the reaction chamber and a drain for the inert gas both so arranged that the inert gas a flows through the liquid contained in the reaction chamber, or a drive for giving the reaction chamber a motion.

The analyzer can further comprise a magnet, which by means of the control electronics is so orientable relative to the reaction chamber that magnetic or paramagnetic particles contained in the reaction chamber are transported by magnetic force into a region of the reaction chamber, in which a fluid line attached to the reaction chamber connects the reaction chamber fluidically with the first microfluidics unit. In a possible alternative embodiment, the analyzer can have a switchable electromagnet, which is so arranged relative to the reaction chamber that magnetic or paramagnetic particles contained in the reaction chamber are transported by magnetic force into a region of the reaction chamber, in which a fluid line attached to the reaction chamber connects the reaction chamber fluidically with the first microfluidics unit. The control electronics can be embodied to switch the electromagnet.

The first and second microfluidics units can be arranged together in a cartridge, especially a replaceable cartridge. Alternatively, the first microfluidics unit can be arranged in a first cartridge arranged and the second microfluidics unit arranged in a second cartridge different from the first cartridge, wherein the first and the second cartridges are connectable fluidically with one another, in order to transport liquid from the first cartridge into the second cartridge. The first and second cartridges can be embodied replaceably.

The first microfluidics unit can be adapted to receive the particles with bound biomolecules and to contact them with one or more lysis reagents, in order to release nucleic acid of the adsorbed biomolecules, then to bind the released nucleic acids to the particles and to wash the particles with the bound nucleic acids one or more times with a wash solution. The control electronics can be adapted to control a transport of the particles and optionally the reagents through the first microfluidics unit.

The first microfluidics unit can be adapted further to remove the nucleic acids from the particles by elution and to transport the eluate into the second microfluidics unit via a fluid line connecting the first microfluidics unit with the second microfluidics unit. The control electronics can be adapted to control the elution and the transport of the eluate.

In an alternative embodiment, the first microfluidics unit can be adapted to receive the particles with bound biomolecules and thermally release the biomolecules bound to the particles or the nucleic acids contained in the biomolecules in a buffer solution, for example, in a phosphate buffered, salt solution (PBS buffer), in water or in a tris buffer, which optionally contains a complexing reagent, e.g., a chelate former such as EDTA, wherein the control electronics is adapted to control the thermal releasing of the biomolecules and to transport the buffer solution with the biomolecules, or nucleic acids, dissolved therein into the second microfluidics unit for subsequent amplification. This embodiment is suitable for use, for example, in an application, in which the biological target substance to be detected is a nucleic acid present released in the liquid.

The first microfluidics unit can contain reaction components in solid form required for releasing and/or isolating the nucleic acid, for example, in the form of pellets won through lyophilization, or in reagent chambers closed by microvalves or in reagent packs closed by films, which can be opened automatically by force or heat effect, especially by means of the control electronics.

Reagents for the amplification can be held in corresponding manner in the second microfluidics unit, which can have one or more detection chambers, in which can be performed the amplifying of a target nucleic acid or in parallel a plurality of different target nucleic acids. The second microfluidics unit can include a temperature control system for the detection chambers. The temperature control system can have, for example, thermoelectric elements controllable by the control electronics.

The control electronics can involve an electronic data processing system having at least one processor and a memory, in which one or more operating programs are stored, which are executable by means of the processor, in order automatically to control the analyzer and in order to obtain qualitative or quantitative, analytical results from the measurement signals of the sensor. The operating programs can be so embodied that the control electronics controls the analyzer to execute the above-described method. The control electronics can, moreover, have a user interface, e.g., a touch screen or other display in combination with an input keypad. The control electronics can be adapted to be connected for communication wirelessly by radio or via a data link with another data processing system, e.g., a computer, a process control, and/or an, especially portable, display or service device.

The analyzer can, thus, perform online determinations of biological target substances in a liquid, without requiring manual sample taking or preparation or the transport of samples to a laboratory. The analyzer can especially execute sample taking and detection cycles completely automatically, and these can be executed e.g., by the control electronics event triggered or at predetermined time intervals.

The invention will now be described in the following based on examples of embodiments illustrated in the figures of the appended drawing. In such case, equal reference characters refer to equal components of the devices shown in the figures. The figures of the drawings show as follows:

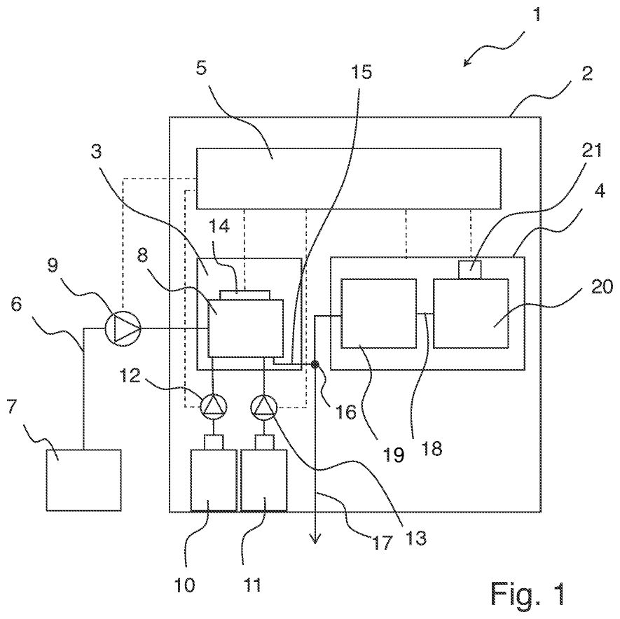

FIG. 1 a schematic view of an online analyzer for qualitative or quantitative determining of a biological target substance in a liquid; and

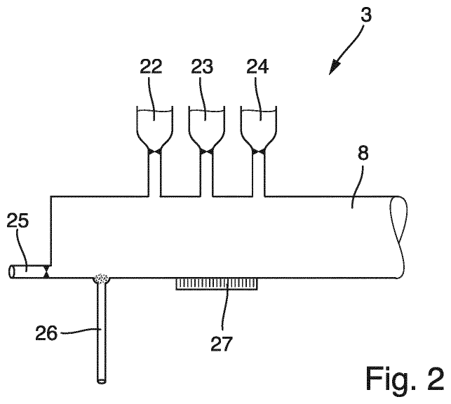

FIG. 2 an example of an embodiment of a process unit of the online analyzer shown in FIG. 1.

FIG. 1 shows schematically an example of an embodiment of an online analyzer 1 according to the invention for detection of a biological target substance in a liquid. Analyzer 1 includes in the present example a housing 2, in which all components of the device are accommodated. These components comprise in particular a process unit 3, a detection unit 4 and a control electronics 5. Process unit 3 forms the core of the invention, since it is adapted to concentrate biological target molecules in a volume of a liquid to be analyzed, and for detection and, in given cases, preparatory steps, such as extraction and/or isolation of nucleic acids, and to forward concentrate to the detection unit 4.

Process unit 3 is fluidically connected via a sample supply line 6 with a sample supply 7 or with a process container, e.g., a pipeline or a reactor or a fermenter, in which the liquid to be analyzed is contained. Serving for transport and metering of the liquid from the sample supply 7 into a reaction chamber 8 contained in the process unit 3 is a pump 9, which is controllable by the control electronics 5 according to an operating program stored in the electronics 5 and executed by such. The reaction chamber 8 is also connectable fluidically with reaction component reservoirs 10, 11. Contained in the reaction component reservoirs 10, 11 are reagents, which assist the concentrating of biomolecules contained in the liquid. Each reaction component reservoir 10, 11 in the present example is connected via a fluid line fluidically with the reaction chamber in the process unit 3. Serving for transport of the reaction components through the fluid lines are pumps 12, 13. It is also possible that the reaction components are integrated directly in a process unit 3. The transport of the reaction components can, of course, be accomplished with other means known to those skilled in the art, e.g., pneumatically or through exploitation of hydrostatic pressure.

Process unit 3 can optionally have a temperature control unit (not shown in FIG. 1), which serves to set a certain temperature in the reaction chamber 8. The temperature control unit can comprise a resistance heater, a cooling element and/or a thermoelectric element for the optional heating or cooling and be connected with the control electronics 5, wherein the control electronics 5 is adapted to control the temperature in the reaction chamber 8.

Process unit 3 can, moreover, have an apparatus 14 for moving or stirring a liquid, or liquid mixture, contained in the reaction chamber 8. Apparatus 14 includes in the present example a drive for moving, e.g., shaking, the reaction chamber 8. Apparatus 14, especially the drive, can be controlled by the control electronics 5.

Reaction chamber 8 is fluidically connectable via the fluid line 15 selectively with the detection unit 4 and with a liquid discharge 17 by means of a valve 16 actuatable by the control unit 5.

Detection unit 4 includes in the present example an analysis cartridge, in which are integrated both microfluidics units fluidically connected with one another via the fluid line 18, namely a first microfluidics unit 19 and a second microfluidics unit 20. In another embodiment, the microfluidics units can also be accommodated in separate cartridges. The analysis cartridge is replaceable in the present example.

The first microfluidics unit 19 is adapted to receive from the reaction chamber a sample containing the concentrated biomolecules and to prepare such for subsequent amplification and detection in the second microfluidics unit 20. Such preparation can occur by means of extraction and isolation methods known per se. For this, the first microfluidics unit 19 includes reagents and optionally a temperature control unit for incubation of reaction mixtures produced in the microfluidics unit 19. For control of transport of the sample from the first microfluidics unit and for performing the extraction and/or isolation of nucleic acids, the detection unit includes suitable means known to those skilled in the art and controllable by the control electronics 5. The second microfluidics unit 20 includes means for amplification, i.e., reagents and temperature control elements, e.g., thermoelectric elements, as well as means for transport and for aliquoting of fluids within the second microfluidics unit 20. For detection of progress of the amplification in manner known per se, e.g., by means of a real time PCR based method, the detection unit includes a sensor 21, which can, for example, perform fluorescence measurements in the second microfluidics unit 20 and which can output measurement signals to the control electronics 5. Control electronics 5 is adapted, based on the measurement signals, to ascertain a qualitative or quantitative, analytical result.

Control electronics 5 can be a central computer unit, e.g., a CPU, with processor and memory and operating programs stored therein. It can also be divided into a plurality of computational units within the analyzer, e.g., the process unit 3 and the detection unit 4 can each have its own on-site electronics connected for communication with a superordinated electronics, wherein the superordinated electronics and the on-site electronics together form the control electronics 5.

FIG. 2 shows the process unit 3 of the online analyzer 1 in detail. The construction of the process unit 3 shown in FIG. 2 is only one possible example of an embodiment. Those skilled in the art can make use of a large number of variants, without departing from the scope of the invention.

Process unit 3 includes a reaction chamber 8 with different inlets and outlets.

The supply lines 22, 23, 24 are connected with supply containers for reagents, which serve as reaction component reservoirs 10, 11. A port 25 is connected with the sample supply line 6 for supplying a certain volume of liquid to be analyzed into the reaction chamber 8. Another port 26 is connected with the fluid line 15, which connects the reaction chamber 8 with a sample discharge as well as with the detection unit 4. Optionally, valves are arranged in the supply lines and actuatable by the control electronics.

Moreover, the process unit 3 in the present example of an embodiment includes a magnet 27 movably arranged on the housing of the reaction chamber 8.

The method steps of the invention running in the process unit 3 take place as follows. In a first step, the control electronics 5 controls the pump 9 for the transport of a first volume of liquid from the sample supply 7 via the sample supply line 6 and the port 25 into the reaction chamber 8. The liquid can be, for example, water from a body of water, from a water supply system or from a water treatment process. Via the supply lines 22, 23 and 24, with control by the control unit 5, reaction components are added to the liquid in the reaction chamber 8. Such can occur during or after introduction of the liquid via the sample supply line 6 into the reaction chamber 8. Added as reaction components in this example of an embodiment are magnetic or paramagnetic particles, an alginate solution and a calcium chloride solution.

The addition of such reaction components enables concentration of biomolecules present in the liquid and preparation for a nucleic acid extraction. The method is basically described in DE 10 215 894 A1. With presence of particles, calcium chloride and alginate, it is accordingly possible to bind biomolecules, e.g., viruses, bacteria or even released nucleic acids in the form of an alginate complex on the particles. In this way, it possible to reduce a large volume water sample to a very much lower sample volume, in such a manner that the biomolecules from the first volume of liquid concentrate on the particles.

Another advantage results from the fact that it becomes possible with the approach of the invention to bind bacteria, viruses, as well as even released nucleic acids completely unselectively on one and the same type of particle. Thus, the particles do not need to be modified, i.e., for example, equipped with an antibody or other ligand, for the concentrating of the target substance to occur. Correspondingly, it is also possible in simple manner with the method of the invention to extract a large number of different biological target substances, firstly, by means of concentration on the particles, and subsequently to make determinations by means of a PCR based method. In the case of methods, which apply particles modified with antibodies or other ligands for concentrating the target substance, a plurality of different particles is required for the parallel determining of a plurality of target substances in a sample. That is significantly more complex and so expensive that its use for continuous monitoring of biological parameters in applications of the process industry would not be cost effective.

In a method variant, the addition of calcium chloride and alginate can be omitted. This variant is advantageously used when the target substance to be detected is a bacterium. Bacteria adsorb nonspecifically on small particles. While this adsorption is not as efficient as the binding in the presence of alginate and calcium ions, nevertheless, in this way, especially the bacteria contained in the liquid can be concentrated on the particles, while released nucleic acids and viruses remain in the liquid and, thus, do not reach the subsequent extraction and analysis steps.

For binding the biomolecules on the particles, they can be dispersed in the liquid, e.g., by means of a stirring apparatus actuatable by the control electronics 5 or by means of a drive (not shown in FIG. 2), which effects a movement of the process unit 3.

The particles with the bound biomolecules are then sedimented and brought together by means of the movable magnet 27. The magnet 27 then moves the particles to the port 26 of the process unit 3. Port 26 is arranged somewhat depressed. The particles are moved by means of the magnet 27 into the depression and gathered there.

In an alternative embodiment, the reaction chamber 8 can be moved relative to the magnet. It is also possible that the magnet is a switchable electromagnet, which is arranged in the region of the port 26, and is switched on by the control electronics 5, in order to pull the particles into the region of the port 26 by means of the magnetic field. Alternatively, instead of the magnet, a pin (which can likewise be embodied magnetically) or a pipetting apparatus or a combination of magnet, pin and/or pipetting apparatus is used for the movement and transfer of the particles.

Port 26 is fluidically connected with the fluid line 15, which is connectable via the valve 16, on the one hand, with the detection unit 4, and, on the other hand, with the liquid discharge 17. Via the fluid line 15, by means of the control electronics 5, the particles are rinsed with a small volume of the liquid contained in the reaction chamber through the fluidics line 15 into the first microfluidics unit 19. For this, another pump can serve, which is controlled by the control electronics 5. Also valve 16 is actuated by the control electronics 5. In the first microfluidics unit 19, an automated nucleic acid extraction and/or isolation occurs, while in the second microfluidics unit 20 downstream from the first microfluidics unit 19, an automated amplification and detection by means of PCR, e.g., real time PCR, occurs. Microfluidics units suitable for the method of the invention for automated nucleic acid extraction, amplification and detection are basically known in the state of the art. Advantageous for the present invention is a centrifugal platform, such as described, for example, in WO 2013/045631 A1 and EP 2 621 632 A1. Possible however also is the application of other microfluidics platforms or lab-on-chip systems, which provide liquid and reagent transport by means of capillary forces, pumps or pneumatic means, instead of centrifugal force.

The extraction and/or isolation of the nucleic acids in the first microfluidics unit 19 occurs by means of known reagents and methods, such as have already been described above. When the biological target substance is not nucleic acids already released in the liquid brought from the sample supply, the biomolecules bound on the particles are lysed. The released nucleic acids are bound back on the particles. Thus, the same particles serve for concentrating the biomolecules in the original sample and also for subsequent isolation of released nucleic acids. The particles with the bound nucleic acids are then washed in known manner, in order to release the nucleic acids finally from the particles. The eluate containing the released nucleic acids is then transported into the second microfluidics unit 20 again under control of the control electronics 5.

In the second microfluidics unit 20, defined volumes of the eluate are transported into one or more detection chambers and amplification reagents added. In the detection chambers, amplification occurs, whose progress is monitored by means of florescence measurements with the sensor 21. The sensor 21 outputs measurement signals to the control electronics 5. This ascertains from the measurement signals a qualitative value, which tells whether the certain biological target substance is contained in the liquid removed from the sample supply 7. Alternatively or supplementally, the control electronics 5 can ascertain from the measurement signals also a quantitative value, which provides a concentration of the biological target substance in the liquid. In an advantageous embodiment, mutually differing target nucleic acids can be amplified in a plurality of detection chambers, and so, in parallel, a large number of different biological target substances can be determined qualitatively or quantitatively in the liquid.

Before or after transfer of the particles from the reaction chamber 8 to the detection unit 4 or after detection has occurred, the residual liquid in the reaction chamber 8 is removed from the process unit 3 through the liquid discharge 17. Process unit 3 can be rinsed sufficiently with a washing, or rinsing, medium, especially with liquid from the sample supply 7, and the process can then be repeated for another measuring. The microfluidic units 19, 20 in the detection unit can be replaced with new microfluidics units 19, 20 for the other measuring.

In an alternative, especially advantageous method embodiment, when the biological target substance in the liquid is present at very low concentration, there is the option that, after the concentrating of the biomolecules contained in the liquid by binding to the particles, the liquid is removed from the process unit 3, but the particles are not yet transferred for subsequent nucleic acid extraction in the detection unit 4, but, instead, remain in the process unit 3. Then another, second volume of liquid can be introduced into the reaction chamber 8 from the sample supply 7. The particles are re-suspended in the second volume of liquid and there occurs the renewed addition of alginate solution and calcium chloride. In this way, a cumulative process is started, which enables utilization of a greater liquid volume and therewith, in given cases, also an increasing of the sensitivity. Such pass-throughs can be repeated a plurality of times.

### Illustrative Example: Detection of Bacteria and Free DNA in a Water Sample

A starting sample was surface river water from the Havel River. Samples were spiked with bacteria (salmonella) and a released, synthetic DNA (control-fragment DNA).

A sample volume of 50 ml of the water was transferred into an experimentally produced process unit 3 via a port 26. To the sample was added via a port 24 a volume of 100 μl from a reservoir of an alginate solution. Process unit 3 was tilted back and forth for short time for the purpose of mixing. Then a volume of 100 μl of a calcium chloride solution was added from a reservoir to the sample via a port 23 and the process unit was tilted anew. Via another port 22, 50 μl of paramagnetic particles (MAG Suspension; AJ Innuscreen GmbH) were added to the sample from a reservoir and the process unit 3 tilted anew. After an incubation time of 20 min, the particles were collected with the movable magnet 27 and transported to the depression at the outlet port 26 and staged there. Via the outlet port 26, the reaction chamber 8 was then emptied. After the emptying, the collected particles were removed from the process unit via the outlet port 26 with a remainder of the water sample (about 200 μl) still in the reaction chamber.

From here according to the invention, the particles were transported into the detection unit 4 for extraction and further detection. There, extraction of the nucleic acid occurred and subsequently the specific target detection.

The entire process unit 3 is subsequently rinsed with water via the inlet port 25 and outlet port 26 and can subsequently be filled with a new sample for the next measuring.

In this example of an embodiment, the extraction and detection was manually performed, in order to establish that it is possible by means of the method of the invention to convert a large sample volume in an online process into a small sample volume and therewith to enable detection of biological target substances. The extraction was performed as follows. The removed particles were mixed with a lysis buffer (Lysis Solution RL; AJ Innuscreen GmbH) and Proteinase K. After an incubation of 20 minutes at 60° C., a binding buffer was added (Binding Solution SBS; AJ Innuscreen GmbH). This binding buffer in combination with the lysis buffer leads to binding of the DNA released from the bacteria and the free control-fragment DNA on the particles.

The batch was mixed for a short period of time and incubated for 2 minutes. Then, the magnetic particles were separated by means of a magnet. The supernatant liquid was poured off and the magnetic particles were washed with 1 ml of a wash buffer (Washing Solution HS; AJ Innuscreen GmbH). After renewed separation of the magnetic particles, the supernatant liquid was completely removed, and the magnetic particles washed another two times with 80% ethanol. After ethanol removal, elution of the bound DNA occurred by the addition of 100 μl RNase free water and an incubation at 60° C. for 5 minutes. After separation of the magnetic particles, the supernatant liquid was removed and sent to a real time PCR device for following detection of the salmonella DNA and the control-fragment DNA. The results of the real time PCR are set forth in the following table.

TABLE 1
RESULTS OF THE REAL TIME PCR

| | Result Salmonella | Result Control- |
|---|---|---|
| Sample | Detection (CT value) | Fragment (CT value) |
| 1 | 23.1 | 24.7 |
| 2 | 22.5 | 24.5 |
| 3 | 23.3 | 24.5 |
| 4 | 22.8 | 23.9 |

The results show that it is possible to bind from a volume of 50 ml water both bacteria as well as also free DNA on particles, to remove the initial water sample and to limit the particles to a volume of 200 μl. The method occurred in a process unit of the invention. Subsequently, the particles were used for the extraction of the nucleic acid and finally detecting of the bacteria and free DNA by means of real time PCR.

The analyzer and method of the invention thus achieve the object of the invention by bringing together available device systems and concentration technologies in ideal manner and enable the performance of an online liquid analysis of biological targets, which is especially advantageous for monitoring water, e.g., wastewater, of water networks, clarification plants or treatment plants as well as from bodies of water in general. The method of the invention enables, for the first time, without manual intervention, beginning with a large volume sample through to detection, performance of a molecular genetic monitoring of biological targets in practical and economic manner.

1-18. (canceled)

19. A method for automated online detection of at least one biological target substance in a liquid using an online analyzer, the method comprising:
transporting a predetermined first volume of the liquid via a sample supply line into a reaction chamber of a process unit of the online analyzer;
concentrating biomolecules contained in the first volume of liquid by binding to a plurality of particles in the reaction chamber;
transporting the particles with the bound biomolecules into a detection unit of the online analyzer, wherein the detection unit includes a first microfluidics unit and a second microfluidics unit fluidically connectable with the first microfluidics unit;
releasing and/or isolating nucleic acids from the biomolecules bound on the particles;
transporting an eluate comprising the released and/or isolated nucleic acids into the second microfluidics unit;
amplifying at least one target nucleic acid of the eluate in the second microfluidics unit;
registering a measurement signal, which represents a measured variable dependent on progress of the amplifying and/or on a number of copies of the at least one target nucleic acid in the second microfluidics unit; and
qualitatively and/or quantitatively determining the biological target substance in the sample based on the registered measurement signal using a control electronics of the analyzer.

20. The method according to claim 19, wherein, after the concentrating of biomolecules contained in the first volume of liquid, the first volume of liquid is removed from the reaction chamber, a second volume of the liquid is introduced into the reaction chamber, and biomolecules contained in the second volume of liquid are concentrated in the reaction chamber by binding to the particles remaining in the reaction chamber.

21. The method according to claim 19, wherein the particles are magnetic or paramagnetic particles, to which the biomolecules bind unselectively.

22. The method according to claim 19, wherein the concentrating of biomolecules contained in the liquid includes introducing an alginate solution and a salt of a divalent or polyvalent cation, or of an acid, into the reaction chamber, wherein an alginate gel-biomolecule complex forms on the particles.

23. The method according to claim 21, wherein the transporting of the particles with the bound biomolecules into the detection unit comprises:
transporting the particles using at least one of: a magnet movable relative to the reaction chamber; a pin; a magnetic pin; and a pipetting apparatus, into a region of the reaction chamber where a fluid line connects the reaction chamber fluidically with the first microfluidics unit; and
rinsing the particles through the fluid line into the first microfluidics unit.

24. The method according to claim 19, wherein the releasing and/or isolating of nucleic acids from the biomolecules bound on the particles in the first microfluidics unit comprises:
contacting the particles with the bound biomolecules with one or more lysis reagents so as to release nucleic acids of the adsorbed biomolecules;
binding the released nucleic acids on the particles; and
washing the particles with the bound nucleic acids with a wash solution.

25. The method according to claim 24, wherein the method further comprises:
releasing the nucleic acids from the particles by eluting; and
transporting an eluate comprising the nucleic acids into the second microfluidics unit for subsequent amplification.

26. The method according to claim 19, wherein the releasing and/or isolating of nucleic acids from the biomolecules bound on the particles in the first microfluidics unit comprises:
thermal releasing of the biomolecules bound on the particles, or of nucleic acids contained in the biomolecules, in one of water, a tris buffer, or a buffer solution, which buffer solution comprises a phosphate-buffered, salt solution; and
transporting the buffer solution with the biomolecules or nucleic acid dissolved therein into the second microfluidics unit for subsequent amplification.

27. The method according to claim 19, wherein the control electronics of the online analyzer executes all method steps automatically.

28. An online analyzer for detection of at least one biological target substance in a liquid according to the method of claim 19, the online analyzer comprising:
a control electronics; a process unit; and a detection unit,
wherein the process unit includes at least one reaction chamber and a sample supply line connected with the at least one reaction chamber, and wherein the process unit is adapted to receive a predetermined volume of the liquid in the at least one reaction chamber and to concentrate biomolecules contained in the liquid by binding to particles,
wherein the detection unit includes:
fluidically connectable with the reaction chamber, a first microfluidics unit, which is adapted to release and/or to isolate nucleic acids from the biomolecules bound on the particles;
connected with the first microfluidics unit, a second microfluidics unit, which is adapted to receive an eluate comprising the released and/or isolated nucleic acids and to amplify a target nucleic acid; and
a sensor adapted to produce a measurement signal dependent on progress of an amplification performed in the second microfluidics unit and/or on a number of copies of the target nucleic acid in the second microfluidics unit and to output said measurement signal to the control electronics, and
wherein the control electronics is adapted to determine the biological target substance in the liquid, qualitatively and/or quantitatively, based on the measurement signal output from the sensor.

29. The online analyzer according to claim 28, further comprising a sample pump, wherein the control electronics is adapted to control the sample pump for transporting a predetermined volume of liquid into the reaction chamber.

30. The online analyzer according to claim 28, further comprising:
at least one reaction component reservoir, which contains a reaction component for the concentrating of the biomolecules in the reaction chamber, wherein the reaction component comprises at least one of: an alginate solution, a salt of a divalent or polyvalent cation, a salt of an acid, and magnetic or paramagnetic particles; and
a reaction components supply line connecting the at least one reaction component reservoir with the reaction chamber, wherein the control electronics is adapted to dose, or meter, a predeterminable amount of the reaction component into the reaction chamber.

31. The online analyzer according to claim 28, further comprising means for stirring the sample contained in the reaction chamber.

32. The online analyzer according to claim 28, further comprising a magnet, which is so orientable relative to the reaction chamber via the control electronics that magnetic or paramagnetic particles contained in the reaction chamber are transported by a magnetic force of the magnet into a region of the reaction chamber, in which region a fluid line attached to the reaction chamber connects the reaction chamber fluidically with the first microfluidics unit.

33. The online analyzer according to claim 28, wherein the first microfluidics unit and the second microfluidics unit are disposed together in a replaceable cartridge.

34. The online analyzer according to claim 28, wherein the first microfluidics unit is adapted to receive the particles with bound biomolecules and to contact the particles with one or more lysis reagents so as to release nucleic acid of the adsorbed biomolecules, then to bind the released nucleic acids to the particles, and to wash the particles with the bound nucleic acids one or more times with a wash solution, and
wherein the control electronics is adapted to control a transport of the particles and reagents through the first microfluidics unit.

35. The online analyzer according to claim 34, wherein the first microfluidics unit is adapted further to remove the nucleic acids from the particles by elution and to transport the eluate into the second microfluidics unit via a fluid line connecting the first microfluidics unit with the second microfluidics unit, and
wherein the control electronics is adapted to control the elution and the transport of the eluate.

36. The online analyzer according to claim 28, wherein the first microfluidics unit is configured to receive the particles with bound biomolecules and to thermally release the biomolecules bound to the particles, or release the nucleic acids contained in the biomolecules, in water, in a tris buffer, or a buffer solution, which includes a phosphate-buffered, salt solution; and
wherein the control electronics is configured to control the thermal releasing of the biomolecules and to transport the buffer solution with the biomolecules, or nucleic acids, dissolved therein into the second microfluidics unit for subsequent amplification.

37. The online analyzer according to claim 36, wherein the tris buffer includes a complexing reagent.

38. The online analyzer according to claim 37, wherein the complexing reagent is selected for complexing divalent or polyvalent cations bound on the particles with the biomolecules.
